(19) United States
(12) Patent Application Publication
(10) Pub. No.: US 2005/0203047 A1
(43) Pub. Date: Sep. 15, 2005

(54) Delivery vectors for short interfering RNA, micro-RNA and antisense RNA

(76) Inventors: Ulrich Thomann, Stow, MA (US); Nicole Stange-Thomann, Stow, MA (US)

Correspondence Address: Nina L. Pearlmutter, Ph.D. Esq.;Nashoba Legal, P.O. Box 4004, Westford, MA, 01886, US

(21) Appl. No.: 11/076,471

(22) Filed: Mar. 9, 2005

Related U.S. Application Data
(60) Provisional application No. 60/551,921, filed on Mar. 10, 2004.

(51) Int. Cl. A61K 48/00; C12Q 1/68; C07H 21/02; C12N 15/85

(52) U.S. Cl. 514/44; 435/455; 435/6; 536/23.1

(57) ABSTRACT

This invention relates to compositions and methods for transcription and expression of nucleic acids into organisms. In particular, the invention comprises a tRNA vector system to deliver and express short interfering nucleic acid, small interfering (siRNA) and micro RNA (miRNA) and antisense (asRNA) into an organism with high efficiency. The compositions further provide expression of nucleic acids to perform as therapeutic compounds in organisms.

### RELATED APPLICATION(S)

This application claims the benefit of U.S. Provisional Application Ser. No. 60/551,921, filed Mar. 10, 2004, the contents of which are hereby incorporated in their entirety by reference herein.

### REFERENCE TO SEQUENCE LISTING

Incorporated herein by reference in its entirety is a sequence listing, comprising SEQ ID NO:1 to SEQ ID NO:16, contained on a CD-R, filed as three identical copies of an ASCII referenced as “020-0501 US.ST25”, created on Mar. 9, 2005, in MS Windows 98 format and 4.21 KB in size.

### FIELD OF THE INVENTION

This invention relates generally to the field of genetics, genomics and molecular biology. The invention relates to methods and materials used to modulate gene expression in a variety of applications, including use in therapeutic, diagnostic, target validation, and genomic discovery applications thereof. This invention also relates to small nucleic acid molecules, such as short interfering nucleic acid, short interfering RNA, double-stranded RNA, microRNA, antisense RNA and other molecules capable of mediating RNA interference against target nucleic acid sequences. The invention also provides nucleic acids, proteins, cloning vectors, expression vectors, transformed hosts, methods of developing pharmaceutical compositions, methods of identifying molecules involved in physiological development in mammals, and methods of diagnosing and treating diseases of mammals.

### BACKGROUND OF THE INVENTION

Small interfering RNA (siRNA) and micro-RNAs (miRNA) are short double stranded ribonucleic acids that are found in a number of organisms (Review: Dykxhoorn et al. 2003 Nature Reviews Mol Cell Biol 4:457-466). Discovered originally in the nematode Caenorhabditis elegans by Mello and co-workers, some of these RNAs are known to have regulatory or protective function and are encoded in the genome (Fire et al. 1998 Nature 391:806-811). It has since been shown that siRNA-like gene silencing mechanisms, also referred to as post-transcriptional gene silencing, can be functional in virtually any species, including in humans and the mammalian model organism for the biomedical industry, the mouse. Many known miRNA sequences and their position in genomes or chromosomes can be found in http://www.sanger.ac.uk/Software/Rfam/mirna/help/summary.shtml. This resource presently lists 176 known human miRNAs (February 2004) with some of their respective target genes known.

Until recently, when siRNA mechanisms were discovered, antisense RNA (asRNA) was the preferred method to down-regulate genes for functional studies such as target validation and pathway analysis. However, design (sequence choice), synthesis of the RNA molecules, delivery and regulation, prevented asRNA to being used significantly beyond in vitro studies. Several of these problems also directly translate into the siRNA field (see below).

Antisense RNA in principle, and siRNA and miRNA can be expressed from gene constructs, but only siRNA and miRNA molecules are also encoded naturally in the genomes of many species. Antisense RNA usually comprises a synthetic single-stranded nucleic acid that can bind to its target by forming base pairs.

Although there are some differences in the expression and maturation of siRNAs and miRNAs, the final and active product is in both cases a short, 19-22 nucleotide long, double-stranded RNA molecule (reviewed in Dykxhoorn et al. 2003 Nature Reviews Mol Cell Biol 4:457-466 and Steinberg 2003 Scientist June 16:22-24). Active siRNA is typically formed by two 21-23 nucleotide long ribo-oligonucleotides that form a 19 base pair long duplex with symmetric 2-3 nucleotide long terminal overhangs having 5′phosphate and 3′ hydroxyl groups. As a result of nucleotide sequence homology of siRNA to their cellular target RNA, one strand, usually the antisense strand binds to the target and renders it inactive and ‘flagged’ for degradation. Thus, in theory, as is the case for antisenseRNA, the antisense strand of siRNA and mciroRNA is sufficient for an inhibitory activity, but the double-stranded nature may be necessary for stability and cellular transport.

Although naturally occurring siRNA was detected more than 5 years ago, only recently have researchers attempted to apply this mechanism of action in genomic research for drug discovery and development. siRNAs can be engineered to bind or recognize virtually any RNA target in the cell and thus be used as “knock-down” tools to silence or down-regulate gene expression through RNA inactivation. The same is becoming obvious for miRNAs.

While this use of siRNA for basic research is a growing field, its postulated utility, being useful as a therapeutic, has so far been less successful. This is not due to a lack of specificity but to the problem of targeted delivery of the active nucleic acid—the RNA molecule itself—into an organism. Thus the same reasons that prevented antisense RNA from being widely used as therapeutic, ie., expensive synthesis and purification, lack of sufficient stability in the cell and thus efficacy, are also the main obstacles for the application of siRNA and microRNA beyond research tools in functional genomics. Since its discovery, “asRNA” or antisense oligonucleotides are usually synthesized as deoxy-oligonucleotides or derivatives thereof (Zemecnik and Stephenson 1978 Proc Natl Acad Sci USA 75:280-284). The use of asRNA, i.e. ribo-oligonucleotides, is impractical, as the stability of these short RNA molecules could not be guaranteed once they are introduced (transfected) into a cell's cytoplasm. Up to date no delivery system exists that would stably express an antisense oligonucleotide from a gene construct, thus eliminating the need for costly synthesis and purification of deoxy-oligonulceotide derivatives for direct transfection.

A possible solution to this direct delivery of RNAi molecules (siRNA, miRNA, asRNA) is to encode these sequences in gene constructs and transform cells with the resulting recombinant DNA molecules. The genes are transcribed by the cell's transcription machinery and thus serve as internal source of siRNA, microRNA, or asRNA. This principle is widely used beyond the RNAi field, such as in transient and stable transformation of cells, in creating transgenic organisms and in expression of natural or engineered proteins or RNAs in host cells.

One problem so far prevents the use of DNA vectors as a tool for using siRNA or microRNA as a therapeutic in organisms. Isolated cells in culture can be transformed with nucleic acids such as plasmids, DNA or RNA without triggering any adverse reaction by the host. However, cells or tissue of a multicellular organism usually recognize any foreign genetic material and react defensively through the organism's immune system. Thus, it is virtually impossible to deliver siRNA encoded in a larger construct to a multicellular organism such as a mammal without inducing an unwanted adverse event. Direct delivery of plasmids or a linear double stranded DNA encoding gene(s) for non-host sequences resulting in transcripts (RNA molecule longer than 200 bp or so) to a mammalian organism can result in a dramatic increase in interferon concentrations that may in turn trigger destruction of the foreign DNA or RNA before the RNA is sufficiently expressed and can reach its intended target or assert function. In other cases the cell or tissue that was transformed and is expressing the foreign gene maybe undergoing apoptosis. This mechanism is a natural defense against foreign genetic material introduced by a number of pathogens such as viruses, bacteria etc.

Three leading methods are currently used to deliver DNA or RNA constructs into cells or tissue: the hydrodynamic method which has been published on by McCaffrey (Nature 2002 418:38-9), Lewis (Nature Genetics 2002 32:107-8), and E. Song (Nature Medicine 2003 9:347-51); lipid-based delivery, which has been reported by Sorenson (J Mol Biol 2003 327:761-6); and naked siRNA, which Makimura and colleagues described in a recent article (BMC Neurosci. 2002 3:18). Of these, the hydrodynamic delivery produces better results but would have no clinical significance as delivery of the RNA or DNA in question cannot be targeted towards a specific tissue and the method is used for isolated cells only.

A very promising method of in vivo targeted delivery of nucleic acid molecules or expression constructs (fragments/plasmids) to cells or tissue is by electrical stimulation or electroporation. However, this method still demands accessibility of the tissues/cells to be transformed with a gene construct or naked RNAi molecule. So far electroporation-mediated delivery in vivo was used to target skin (murine melanoma; Rols et al., Nat Biotechnol 1998 16;168) muscle tissue (Kishida et al,. J Gene Med 2004 6:105), eye (retina; Matsuda and Cepko, Proc Natl Acad Sci USA 2004 101:16) and brain (Akaneya et al. J Neurophysiol 2005 93:594). Direct delivery of RNAi cloned into delivery vectors as demonstrated by this invention to skin tissue or joint interstitial fluid, presents a unique opportunity to prove RNAi's potential as therapeutic for example for treatment of pain and inflammation, rheumatoid arthritis and skin cancers (see Rols et al. 1998).

A small siRNA, miRNA or asRNA molecule itself will not induce an interferon response. However, as stated above, delivering such constructs directly is costly due to the expense of synthesis and purification of the required large concentrations of ribo-oligonucleotides. Furthermore, such constructs lack adequate stability in cellular environments and cannot be sufficiently controlled as to their timely activation and expression in the target tissue. Only lower eukaryotes such as worms, plants and fungi can replicate siRNA molecules. Higher eukaryotes such as rodents, humans, etc. lack this ability so that direct siRNA delivery therefore is at best transient (Zamore 2002 Science 296:1265-1269). In these species, a DNA-vector mediated delivery and/or expression system is preferred.

### SUMMARY OF THE INVENTION

This invention describes a novel vector system that can deliver and express small interfering RNA (siRNA), micro RNA (miRNA) and antisense RNA (asRNA) constructs into an organism without inducing a cellular immune reaction such as interferon production and/or an inflammatory response. The delivery system can be used for transforming small interfering and micro RNAs into therapeutics either by using a DNA construct expressing a small interfering RNA or micro RNA construct or by directly delivering the construct as RNA. In fact, this system can be used to deliver any short oligonucleotide or short gene or functional coding sequence.

This invention further provides a method of modulating gene expression through RNA interference comprising: incorporating at least one oligonucleotide encoding a short interfering nucleic acid molecule, including coding sequence for RNAi, microRNA and/or antisense RNA into a tRNA gene construct; and/or transforming a host cell with the modified tRNA gene construct under conditions wherein the short interfering nucleic acid is expressed and cleaved or spliced from the expressed tRNA molecule so that the short interfering nucleic acid can enter the cytoplasm and interact with its target host molecule. Additionally, the invention provides a method of modulating gene expression through RNA interference comprising: replacing all or part of an intron of a mammalian tRNA gene encoding tyrosine (tRNA^{Tyr}) or other intron-encoding tRNA gene with at least one oligonucleotide encoding a short interfering nucleic acid; and/or transforming a mammalian host cell with the modified tRNA gene construct under conditions wherein the short interfering nucleic acid is expressed and spliced from the expressed tRNA^{Tyr} or other intron-containing tRNA molecule so that the short interfering nucleic acid may enter the cytoplasm and interact with its target host molecule. The mammalian host cell can be a mouse, rat, primate or human cell. This invention also encompasses the above methods for any animal (vertebrate or invertebrate) or plant cell.

In a further embodiment, this invention includes a method of modulating gene expression through RNA interference comprising producing a modified tRNA gene construct by: fusing a nucleic acid encoding the 5′ end of the lower (inhibitory) strand of a double-stranded siRNA to the nucleic acid encoding a tRNA gene construct at the site wherein the 3′ end of the first tRNA exon is encoded; connecting the nucleic acid encoding the 3′ end of the lower strand of the siRNA with nucleic acid encoding a three to six base pair spacer to nucleic acid encoding the 5′ end of the upper strand of the siRNA; fusing nucleic acid encoding the 3′ end of the upper strand of the siRNA to nucleic acid encoding the 5′ end of the second exon of the tRNA; and transforming a host cell with the modified tRNA gene construct under conditions wherein the short interfering nucleic acid is expressed and spliced from the modified tRNA construct or expressed tRNA from the transcribed modified tRNA construct so that the short interfering nucleic acid can enter the cytoplasm and interact with its target host molecule.

This invention further includes a short interfering nucleic acid molecule, comprising a coding sequence for siRNA, miRNA or antisense RNA incorporated into a tRNA gene construct. The coding sequence of the short interfering nucleic acid molecule can be incorporated into a tRNA gene construct by fusion of the 3′ end of the coding sequence to the 5′ end of the tRNA gene, or it can be incorporated into a tRNA gene construct by fusion of the 5′ end of the coding sequence to the 3′ end of the nucleic acid encoding the tRNA gene. Further, the coding sequence can be incorporated into a tRNA gene construct by fusion of the 5′ end of the coding sequence to the 3′ end of the nucleic acid encoding the first exon of tRNA gene and fusion of the 3′ end of the coding sequence to the 5′ end of the nucleic acid encoding the second exon of the tRNA gene. In another aspect, incorporation can take place in the reverse wherein the coding sequence is incorporated into a tRNA gene construct by fusion of the 3′ end of the coding sequence to the 5′ end of the nucleic acid encoding the first exon of tRNA gene and fusion of the 5′ end of the coding sequence to the 3′ end of the nucleic acid encoding the second exon of the tRNA gene. The tRNA gene constructs described above can comprise nucleic acids encoding siRNA, miRNA or antisense RNA (asRNA). Preferably, a tRNA gene construct will encode 19-30 nucleotides of a sense strand of an siRNA and/or 19-30 nucleotides of an antisense strand of the siRNA.

The use of a naturally-occurring (genome-encoded) intron-containing tRNA gene makes it possible to deliver, express and release three independent RNAi molecules (siRNA, microRNA or asRNA) in one construct. These can either be used to target three independent genes for silencing or to increase the gene silencing effect by targeting the same gene with two or three RNAi molecules that bind to different sequence motifs.

In another aspect of this invention, the modified tRNA constructs described herein can be used to deliver anti-cancer agents, anti-inflammatory or pain-relieving agents, such as siRNA, microRNA, antisense RNA, and other small interfering oligonucleotides systemically or to localized tissues. This invention also comprises a short interfering nucleic acid molecule that can be a coding sequence for siRNAi, miRNA and/or asRNA incorporated into a tRNA gene.

In another aspect of this invention, the use of a naturally-occurring vector to deliver a therapeutic interfering nucleic acid molecule into an organism is exemplified, wherein the interfering nucleic acid molecule is selected from the groups consisting of a siRNAi, miRNA and antisense RNA. Further described is a synthetic tRNA construct that incorporates RNAi sequences in the 5′ leader or 3′ trailer, used for direct transformation of cells or organisms, and a synthetic tRNA construct comprising either miRNA sequence precursors or mature miRNA sequences in either its 5′ leader or 3′ trailer, or in its intron that can be used for direct transformation of cells or organisms. Additionally, a synthetic tRNA construct comprising an antisense RNA sequence in either its 5′ leader and/or 3′ trailer, or its intron can be used for direct transformation of cells or organisms.

This invention also describes the use of a short interfering oligonucleotide, including antisense RNA, transfected into a cell by means of a tRNA vector. This invention can be organized into a kit for transfecting a cell wherein the kit comprises nucleic acid encoding a short interfering RNA or antisense RNA incorporated into a tRNA vector.

Another aspect of this invention provides a method of treating an organism comprising administering a tRNA gene construct modified to incorporate nucleic acid encoding a short interfering RNA, microRNA or antisense RNA. The organism can be an animal or plant. The animal can be a mammal, including a human.

### BRIEF DESCRIPTION OF THE FIGURES

FIG. 1 depicts the primary and secondary structure of a mature tRNA molecule.

FIG. 2 illustrates the transcription of a tRNA gene and subsequent processing of the transcription product, including folding and splicing, into a mature, functional tRNA molecule.

FIG. 3 illustrates transcription and subsequent processing of a modified tRNA gene wherein the 5′ leader and/or 3′ trailer (flanking sequences) of the original tRNA gene are replaced by the coding sequence for siRNA motifs.

FIG. 4 illustrates an example using a siRNA sequence incorporating the coding sequence into a tRNA gene and by depicting its transcription and maturation releasing an active siRNA molecule.

FIG. 5 shows an example of a microRNA-tRNA gene fusion vector.

FIG. 6 shows another embodiment of a siRNA-tRNA fusion vector wherein the intron of the original tRNA gene is replaced by the coding sequence of a siRNA motif.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

This invention overcomes the delivery, stability and adverse reaction problems associated with transforming tissue directly with DNA or RNA constructs that encode siRNA or miRNA or asRNA by fusing the double-stranded construct to transfer RNA (tRNA), a cellular abundant RNA species that is essential for protein biosynthesis. The transfection complexes of this invention provide substantially higher transfection efficiencies than presently available systems as the siRNA molecules are synthesized in situ. Transfer RNA or tRNA refers to a set of genetically encoded RNAs that act during protein synthesis as adaptor molecules, matching individual amino acids to their corresponding codon on a messenger RNA (mRNA). In higher eukaryotes such as mammals, there is at least one tRNA for each of the 20 naturally occurring amino acids.

In eukaryotes, including mammals, tRNAs are encoded by families of genes that are 73 to 150 base pairs long. A complete list of tRNA genes, corresponding tRNAs etc. is given in http://rna.wustl.edu/tRNAdb/. A tRNA gene is transcribed by DNA dependent RNA polymerase III in a very well studied process into a tRNA precursor (pre-tRNA), a tRNA that essentially has additional sequences at its 5′ and 3′ end that must be removed in order to get a biologically active tRNA. These additional sequences are referred to as 5′ leader and 3′ trailer. The maturation of the pre-tRNA to tRNA is dependent on certain conserved sequences and secondary structures of the molecule and at least two enzyme systems that are present in every cell, such as RNase P (Altman S. 2000 Nat Struct Biol 7, 827) and the 3′ processing enzyme RNase Z (Schiffer et al. 2002 EMBO J. 21, 2769; Pellegrini et al. 2003 EMBO J; 22, 4534).

In one embodiment, this method provides a tRNA gene as vector for siRNA delivery by fusing the 3′ end of the coding sequence of the sense (upper) strand of the siRNA to the 5′ end of the structural tRNA gene and the 5′ end of the coding sequence of the antisense (lower, inhibitory) strand of the siRNA to the 3′ end of the structural tRNA gene sequence using short (1-3 bp) sequences as short linkers to preserve the processing enzyme's conserved cleavage sites. It is important to add that the reverse fusion, i.e., by fusing the 3′ end of the sequence of the lower (inhibitory) strand of the siRNA to the 5′ end of the structural tRNA gene and the 5′ end of the upper strand of the siRNA to the 3′ end of the tRNA gene will also result in an active siRNA after 5′ and 3′ maturation.

In another embodiment, this method also provides a tRNA gene as vector for miRNA and/or asRNA by fusing the 5′ end of the coding sequence of a miRNA or asRNA to the 3′ end of a tRNA gene or the 3′ end of the coding sequence to the 5′ end of the tRNA gene.

Because neither pre-tRNA nor tRNA is recognized as foreign to the cells of an organism, no immune response is triggered when such genes are transcribed and recombinant pre-tRNA is synthesized by the cell's own transcription machinery. Upon delivery of the RNAi-tRNA fusion gene into a cell and induction of RNA transcription, the siRNA-tRNA or miRNA-tRNA or asRNA-tRNA will be synthesized in the nucleus (where the necessary transcription machinery is located) and the resulting pre-tRNA molecule will spontaneously fold into the characteristic tRNA cloverleaf structure, including an extended double-stranded sequence comprising the siRNA molecule that is fused to the acceptor stem of the tRNA. The pre-tRNA molecule also can have the sequence of a miRNA or asRNA fused either to its 5′ end or 3′ end. These molecules are in turn recognized by the tRNA processing enzymes in the nucleus and cleaved as specific sites to release a double stranded siRNA molecule or a single stranded miRNA or asRNA and a mature tRNA. Lund and Dahlberg have shown in Xenopus oocytes that only correctly processed mature tRNAs are exported from nuclei in a RanGTP-dependent manner (Dahlberg and Lund 1998 Curr Opin Cell Biol 10:400; Lund and Dahlberg 1998 Science 282:2082), thus leaving the cytoplasm free of any ‘unfamiliar’ RNA molecules that can trigger an anti-inflammatory response such as increase in interferon concentrations. This controlled nuclear release of only mature tRNAs is true of other eukaryotes as well (Sarkar et al. 1999 Proc Natl Acad Sci USA. 96, 14366).

Another embodiment that utilizes tRNAs as vectors for siRNA, miRNA and antisense RNA delivery is through the mechanism of pre-tRNA splicing. The tRNA specific for tyrosine (tRNA^{Tyr}) and certain other tRNA genes in higher eukaryotes are encoded as a gene containing an intron in the anticodon loop of the respective tRNA. The source http://rna.wustl.edu/tRNAdb also provides a comprehensive list of tRNA genes with introns encoded in the human genome and other mammalian genomes. This intron must be removed (spliced) to obtain a functioning tRNA^{Tyr} or other tRNAs. The mechanism is well documented (Stange and Beier 1992 Eur J Biochem 210, 193; van Tol et al. 1987 EMBO J. 6, 35-41) and it is known that the intron sequence itself does not need to be conserved (even introns of over 100 bp in length are accurately removed). Thus the majority of the intron sequence can be replaced by sequences encoding a siRNA, miRNA and/or as RNA.

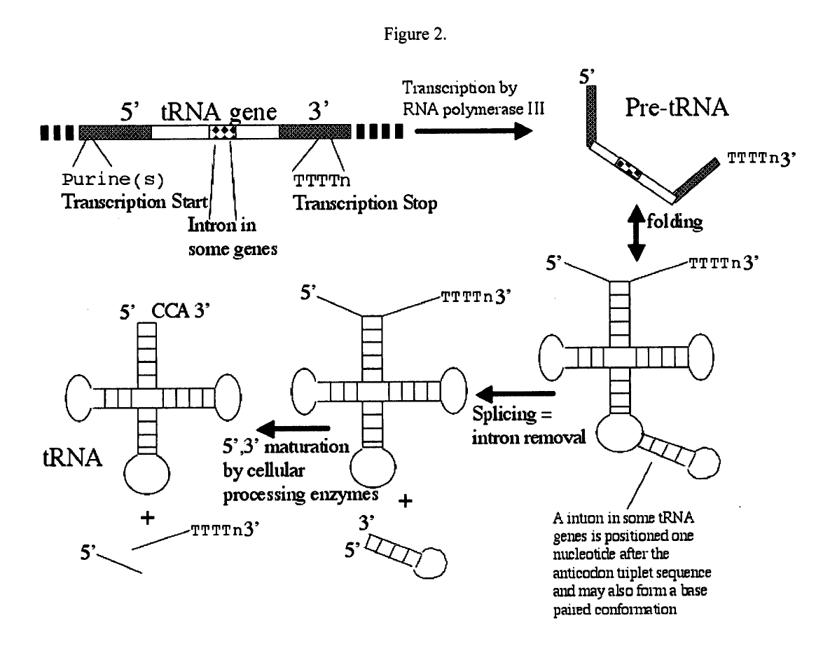

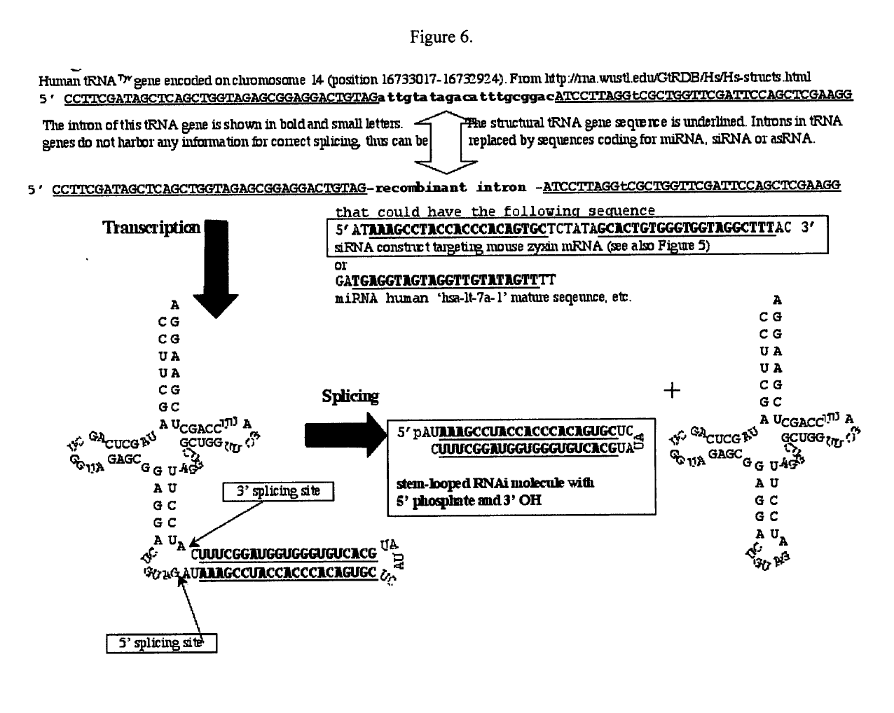

Mammalian siRNA expression plasmids that essentially use a RNA polymerase III promoter as found in genes coding for U6 and H1 RNAs that is placed upstream of the siRNA encoding sequences are available from Upstate USA, Inc. (4588 Collections Center Drive, Chicago, Ill. 60693). Here, the coding sequence for the upper strand and the coding sequnce for the lower strand of the siRNA are separated not by a tRNA sequence but by an unrelated spacer sequence. Upon transcription a RNA hairpin structure is formed with the upper and lower siRNA strand base-pairing. This molecule is then processed by “cellular nuclease” into a functional siRNA. This principle was first described by Myagishi and Taira (2002 Nature Biotechnol 19:497-500) and later adapted by others (Reviewed by Tuschl 2002 Nature Biotechnol 20: 446-448). The short oligo-U sequence that is added at the 3′ end of a siRNA molecule due to the presence and partial transcription of the RNA polymerase III termination signal (FIG. 2) do not change the specificity of the siRNA molecule. A short loop connecting the upper and lower strand of the siRNA molecule, that will also be the product when exchanging a tRNA intron with the coding sequence for a siRNA (FIG. 6), will be processed/cleaved by cellular enzymes, rendering an active siRNA molecule. The vector system might circumvent an interferon response. Kunath et al. (2003 Nature Biotechnol 21:559-561) used it for stable transformation of embryonic stem cell derived embryos inducing a genetic null type by effectively silencing the gene for RasGAP. However, transcribed RNA molecules—including the unprocessed longer precursor molecules—can enter the cytoplasm freely, unlike a pre-tRNA molecule, that is locked or maintained in the nucleus until processed correctly to a mature tRNA, only then releasing the siRNA, miRNA or asRNA molecules simultaneously. Furthermore, in all here described cases that utilize a gene encoding RNAi sequences that are controlled by upstream RNA polymerase III promoters, including Upstate's Mammalian siRNA Expression Plasmids, it is unclear what processing enzymes, if any, are involved to release the mature siRNA molecule and whether an immune response prevents accumulation of the certain transcribed pre-siRNA molecules.

To facilitate an understanding of the invention, a number of terms or phrases are described below:

The term “nucleic acid” is a term of art that refers to a polymer containing at least two nucleotides. “Nucleotides” contain a sugar deoxyribose (DNA) or ribose (RNA), a base, and a phosphate group. Nucleotides are the monomeric units of nucleic acid polymers. Nucleotides are linked together through the phosphate groups to form nucleic acid. A “polynucleotide” is distinguished here from an “oligonucleotide” by containing more than 100 monomeric units; oligonucleotides contain from 2 to 100 nucleotides. “Bases” include purines and pyrimidines, which further include natural compounds adenine, thymine, guanine, cytosine, uracil, inosine, and other natural analogs, and synthetic derivatives of purines and pyrimidines, which include, but are not limited to, modifications which place new reactive groups such as, but not limited to, amines, alcohols, thiols, carboxylates, and alkylhalides. The term nucleic acid includes deoxyribonucleic acid (“DNA”) and ribonucleic acid (“RNA”) and encompasses sequences that include any of the known base analogs of DNA and RNA.

Nucleic acids can be linear, circular, or have higher orders of topology (e.g., supercoiled plasmid DNA). DNA can be in the form of antisense, plasmid DNA, parts of a plasmid DNA, vectors (P1, PAC, BAC, YAC, artificial chromosomes), expression cassettes, chimeric sequences, chromosomal DNA, or derivatives of these groups. RNA can be in the form of oligonucleotide RNA, tRNA (transfer RNA), snRNA (small nuclear RNA), rRNA (ribosomal RNA), mRNA (messenger RNA), antisense RNA, (interfering) double-stranded and single-stranded RNA, ribozymes, chimeric sequences, or derivatives of these groups. “Antisense” is a nucleic acid that interferes with the function of DNA and/or RNA. This can result in suppression of expression. Interfering RNA (“RNAi”) is double stranded short-interfering RNA (siRNA) or single-stranded micro-RNA (miRNA) that results in catalytic degradation of specific mRNAs, and can also be used to lower gene expression.

Nucleic acid can be single (“ssDNA”), double (“dsDNA”), triple (“tsDNA”), or quadruple (“qsDNA”) stranded DNA, and single stranded RNA (“RNA”) or double stranded RNA (“dsRNA”). “Multistranded” nucleic acid contains two or more strands and can be either homogeneous as in double stranded DNA, or heterogeneous, as in DNA/RNA hybrids. Multistranded nucleic acid can be full length multistranded, or partially multistranded. It can further contain several regions with different numbers of nucleic acid strands. Partially single stranded DNA is considered a sub-group of ssDNA and contains one or more single stranded regions as well as one or more multiple stranded regions.

“Preparation of single stranded nucleic acid”: Single stranded nucleic acids can be generated by a variety of means, including denaturation, separation, chemical synthesis, isolation from viruses, enzymatic reaction. “Denaturation” is the process in which multi-stranded nucleic acid is completely or partially separated into single stranded nucleic acids. This can proceed through heating, alkaline treatment, or the addition of chemicals. A mixture of nucleic acids can be “separated” by physical means such as density gradient centrifugation, gel electrophoresis, or affinity purification. Affinity purification can be accomplished by incorporating a ligand in the nucleic acid (e.g., biotin), and using the corresponding ligate (e.g., strepavidin) bound to a matrix (e.g., magnetic beads) to specifically bind and purify this nucleic acid.

“Chemical synthesis” refers to the process where a single stranded nucleic acid is formed by repetitively attaching a nucleotide to the end of an existing nucleic acid. The existing nucleic acid can be a single nucleotide. Single stranded oligonucleotides can be chemically linked together to form long or short nucleic acids.

“Viral” nucleic acids are isolated from viruses.

“Enzymatic reaction” refers to processes mediated by enzymes. One strand of a double stranded nucleic acid can be preferentially degraded into nucleotides using a nuclease. Many ribonucleases are known with specific activity profiles that can be used for such a process. For instance, RNase H can be used to specifically degrade the RNA strand of an RNA-DNA double stranded hybrid nucleic acid, which in itself can have been formed by the enzymatic reaction of reverse transcriptase synthesizing the DNA strand using the RNA strand as the template. Following the introduction of a nick, a nuclease can specifically degrade the strand with the nick, generating a partially single stranded nucleic acid. A RNA or DNA dependent DNA polymerase can synthesize new DNA which can subsequently be isolated (e.g., by denaturation followed by separation). The polymerase chain reaction process can be used to generate nucleic acids. Formation of single stranded nucleic acid can be favored by adding one oligonucleotide primer in excess over the other primer (“asymmetric PCR”). Alternatively, one of the DNA strands formed in the PCR process can be separated from the other (e.g., by using a ligand in one of the primers).

The term “gene” generally refers to a nucleic acid sequence that comprises coding sequences necessary for the production of a therapeutic nucleic acid (e.g., ribozyme) or a polypeptide or precursor. The polypeptide can be encoded by a full-length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as “5′ untranslated sequences.” The sequences that are located 3′ or downstream of the coding region and which are present on the mRNA are referred to as “3′ untranslated sequences.” The term gene encompasses both cDNA and genomic forms of a gene. A genomic form or clone of a gene contains the coding region interrupted with “non-coding sequences” termed “introns” or “intervening regions” or “intervening sequences.” Introns are segments of a gene, which are transcribed into nuclear RNA. Introns can contain regulatory elements such as enhancers, and coding sequences for miRNAs and siRNAs. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term non-coding sequences also refers to other regions of a genomic form of a gene including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotide) or at distant sites (more than 10,000 nucleotides). These non-coding sequences influence the level or rate of transcription and translation of the gene.

The term “isolated” when used in relation to a nucleic acid, as in “an isolated nucleic acid” refers to a nucleic acid sequence that is identified and separated from at least one contaminant nucleic acid with which it is ordinarily associated in its natural source. Isolated nucleic acid is such present in a form or setting that is different from that in which it is found in nature. In contrast, “non-isolated nucleic acids” are nucleic acids, such as DNA and RNA, found in the state they exist in nature.

As used herein, the term “gene expression” refers to the process of converting genetic information encoded in a gene into RNA (e.g., mRNA, rRNA, tRNA, or snRNA) through “transcription” of a deoxyribonucleic gene (e.g., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (ie., RNA or protein), while “down-regulation” or “repression” refers to regulation that decrease production. Molecules (e.g., transcription factors) that are involved in up-regulation or down-regulation are often called “activators” and “repressors,” respectively.

A molecule is “modified,” through a process called “modification,” by a second molecule if the two become bonded through a covalent bond. That is, the two molecules form a covalent bond between an atom form one molecule and an atom from the second molecule resulting in the formation of a new single molecule. A chemical “covalent bond” is an interaction bond, between two atoms in which there is a sharing of electron density.

Delivery of a nucleic acid means to transfer a nucleic acid from a container outside a mammal to near or within the outer cell membrane of a cell in the mammal. The term “transfection” is used herein, in general, as a substitute for the term “delivery,” or, more specifically, the transfer of a nucleic acid from directly outside a cell membrane to within the cell membrane. If the nucleic acid is a primary RNA transcript that is processed into messenger RNA, a ribosome translates the messenger RNA to produce a protein within the cytoplasm. If the nucleic acid is a DNA, it enters the nucleus where it is transcribed into a messenger RNA, tRNA, snRNA or rRNA that is transported into the cytoplasm where it can be translated into a protein if it is a mRNA. Therefore if a nucleic acid expresses its cognate protein, then it must have entered a cell. A protein can subsequently be degraded into peptides, which can be presented to the immune system.

A “therapeutic gene” refers herein to a nucleic acid that may have a therapeutic effect upon transfection into a cell. This effect can be mediated by the nucleic acid itself (e.g., anti-sense nucleic acid), following transcription (e.g., antisense RNA (asRNA), ribozymes, interfering dsRNA or single-stranded RNA), or following expression into a protein.

“Protein” or “peptide” refers herein to homo or hertero condensation polymers of amino acids which are linked by formation of amide bonds from the amino group of one amino acid and the carboxylic acid group of another. These can be a linear series of greater than two amino acid residues connected one to another as in a polypeptide. Proteins or peptides may be fibrous, globular, pigmented by metals, crosslinked, or aggregative.

“Vectors” are nucleic acid molecules originating from a virus, a plasmid, or the cell of an organism into which another nucleic fragment of appropriate size can be integrated without loss of the vectors capacity for self-replication. Vectors can be synthesized. Vectors introduce nucleic acids into host cells, where it can be reproduced. Examples are, plasmids, cosmids, and bacterial and yeast artificial chromosomes. Vectors are often recombinant molecules containing nucleic acid sequences from several sources. Vectors include viruses, for example adenovirus (an icosahedral (20-sided) virus that contains DNA; there are over 40 different adenovirus varieties, some of which cause respiratory disease), adeno-associated virus (AAV, a parvovirus that contains single stranded DNA), or retrovirus (any virus in the family Retroviridae that has RNA as its nucleic acid and uses the enzyme reverse transcriptase to copy its genome into the DNA and integrate into the host cell's chromosome).

The process of delivering a nucleic acid to a cell has been commonly termed transfection or the process of “transfecting” and also it has been termed “transformation.” The term transfecting as used herein refers to the introduction of foreign DNA into cells. The nucleic acid could be used to produce a change in a cell that can be therapeutic. The delivery of nucleic acid for therapeutic and research purposes is commonly called “gene therapy.” The delivery of nucleic acid can lead to modification of the genetic material present in the target cell. The term “stable transfection” or “stably transfected” generally refers to the introduction and integration of foreign nucleic acid into the genome of the transfected cell. The term “stable transfectant” refers to a cell which has stably integrated foreign nucleic acid into the genomic DNA. Stable transfection can also be obtained by using episomal vectors that are replicated during the eukaryotic cell division (e.g., plasmid DNA vectors containing a papilloma virus origin of replication, artificial chromosomes). The term “transient transfection” or “transiently transfected” refers to the introduction of foreign nucleic acid into a cell where the foreign nucleic acid does not integrate into the genome of the transfected cell. The foreign nucleic acid persists in the nucleus of the transfected cell. The foreign nucleic acid is subject to the regulatory controls that govern the expression of endogenous genes in the chromosomes. The term “transient transfectant” refers to a cell, which has taken up foreign nucleic acid but has not integrated this nucleic acid.

The present invention provides a process of delivering a biologically active substance to a target cell by incorporating a nucleic acid encoding an interfering RNA into a tRNA gene construct. The target cell is exposed to the biologically active substance as the tRNA and the interfering RNA are transcribed and the interfering RNA is cleaved or spliced from the mature tRNA. A biologically active substance can be an RNA that interacts with and alters the function of a cell. Where the target cell is located in vitro, the biologically active substance, and the delivery system are typically added to the culture medium in which the cell is being cultured. The active substance and delivery system can be added to the medium either simultaneously or sequentially. Alternatively, the biologically active substance and the delivery system can be formed into a complex and then added to the medium. A complex between a biologically active substance and a delivery system of the present invention can be made by contacting those materials under appropriate reaction conditions. Means for making such complexes are set forth hereinafter in the Example.

Where the target cell is located in vivo, the biologically active substance and the delivery system are typically administered to the organism in such a way as to distribute those materials to the cell. This can be transfection into cells, injections into mammals or invertebrates, or introduction into plant cells via particle gun or other methods. The transfection can also be made tissue-specific by using tissue-specific promoter sequences that are fused to a gene construct to ensure that the gene is only expressed in the tissue of choice. The materials can be administered simultaneously or sequentially as set forth above. In one embodiment, the biologically active substance and the delivery system are administered as a complex. The delivery system and biologically active substance can be infused into the cardiovascular system (e.g., intravenously, intraarterially), injected directly into tissue containing the target cell (e.g., intramuscularly), or administered via other parenteral routes well known to one skilled in the art.

This invention further provides kits containing any of the vectors or transfection complexes described herein. Such kits can further comprise vectors or transfection complexes, instructions, and control materials.

The constructs of this invention can have many uses. Short interfering RNAs can play many roles in vivo. As well as silencing genes such as cancer genes, they can be used for gene expression regulation, transposon silencing, centromeric silencing (e.g, Volpe et al. 2002 Science 297:1833-7), genomic rearrangements (e.g., Yao et al. 2003 Science 300:1581-4), and as an antiviral, antibacterial or antifingal defense.

The constructs of this invention can be used to modify the biochemical and physical activities of both plants and animals. Animals whose activities can be modified include all multicellular animals, including vertebrates and invertebrate animals, such as humans, rodents (mice, rats, guinea pigs, etc.), apes, monkeys, or other primates, as well as birds, reptiles, fish, worms, crustaceans, etc.

Plants that can be modified by this invention include multicellular and unicellular plants; for example, seed plants (dicots or monocots), angiosperms or gymnosperms, vascular or nonvascular plants, ferns, algae (multicellular or unicellular), and the like. In particular, crop plants (C₃ or C₄), such as corn, soybeans, rice, Brassica sp., legumes, sunflowers, sorghum, tomatos, potatos, melons, nuts, fruits and vegetables and the like can be transfected by methods of this invention.

### EXAMPLE ONE

### Cloning and Gene Construction

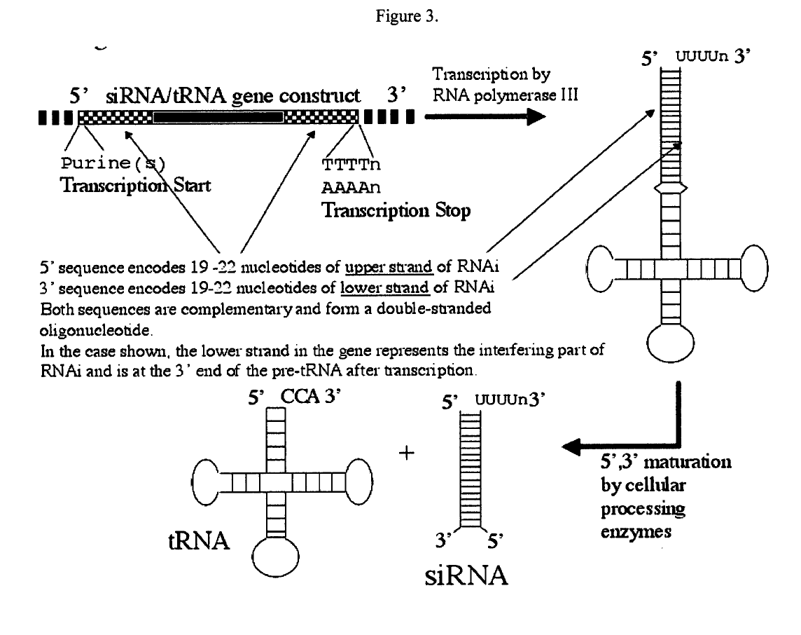

Commercially available components such as primers are used to isolate or PCR-amplify a human tRNA gene starting from genomic DNA. The primers are chimeric so that they add a 5′ and 3′ complementary overhang sequence that encodes a 19-21 nt long double stranded siRNA (see FIGS. 2 and 3). The siRNA sequence can be as long as 30 bp; however, longer sequences can invoke an antiviral response in the organism. The siRNA sequence is designed to partially encode the sequence of a known target gene and maybe selected from commercially available siRNA suppliers. The construct is cloned into a shuttle vector allowing amplification in bacterial systems and also stable transient expression in a mammalian cell or animal model. Injection or transformation of mice through tail vein injection is done as described by Gratsch et al. (2003 Genesis 37:12-7) or Giladi et al. (2003 Mol Ther 8:769-76). Kim et al. (2004 Nature Biotechnol 22:321-325) show that an interferon response can also be triggered and monitored in embryonic kidney cells upon transfection of these cells with siRNA molecules. Interestingly, the authors also show that apparently the 5′ triphosphate, that will be present at siRNA molecules that are externally synthesized via transcription by T7 polymerase (review by Samuel 2004 Nature Biotechnol 22:280-283) is a major trigger of interferon response in cells. No response is triggered when the transfected siRNA contains 5′ phosphates or 5′ hydroxyl groups. This invention provides constructs that lack such 5′ triphosphate triggers.

Dharmacon, Inc. (Lafayette, Colo.; http://www.dharmacon.com/) is providing a list of published siRNA sequences/constructs for gene expression down regulation in mouse, human and other organisms. A public webpage at the Sanger Centre (UK) provides known miRNA precursors as well as the active mature miRNA (http://www.sanger.ac.uk/cgi-bin/Rfam/mirna/browse.pl).

Impact on the target gene (down regulation) is monitored by taking tail tissue or blood from a mouse, that was transfected with a RNAi-tRNA construct cloned into a shuttle vector, and submitting it to gene expression analysis. In the case of human embryonic kidney cells, extracts can be used to monitor RNA and protein levels of the target gene and comparing it to controls (extracts from cells that were transformed with a control vector containing a tRNA construct without an RNAi fusion). In both cases, an interleukin response is monitored via elisa or immunoblot (Western blot)—which can be detected in the medium in cased of cell culture, because interferon is excreted into the extracellular matrix.

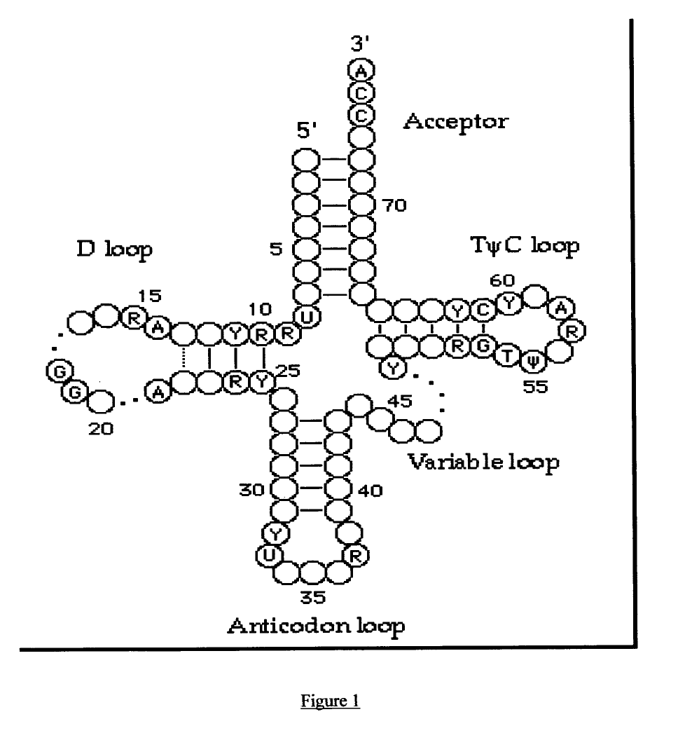

FIG. 1 shows a depiction of the two-dimensional structure of a tRNA molecule. Each circle represents a ribonucleotide. The structure of the mature tRNA has conserved sequences or elements that are important for its function. The conserved ribonucleotides are show by their symbols (within circles) in their structural positions. Explanation of symbols: A=adenine; C=cytosine; G=guanidine; T=thymidine; U=uracil; Y represents C or U; R represents A or G; Ψ represents pseudouridine.

The promoter sequences necessary for RNA polymerase III transcription are encoded in the tRNA gene and thus transcribed into the tRNA and are still deductable from sequences located in the D and TΨC loop. These sequence motifs are sufficient for transcription. Once transcribed, the tRNA folds into its characteristic cloverleaf structure as shown. In reality, the tRNA molecule also has a characteristic three-dimensional structure that is ultimately important for its correct function and recognition by processing enzymes (5′, 3′ processing and splicing as well as addition of the conserved CCA sequence at its mature 3′ end).

As mentioned supra, all promoters necessary for RNA polymerase III transcription are encoded in the structural gene. In rare cases, there are additional sequences upstream (5′) of the gene that have some regulatory function. However, most mammalian genomes encode more than 100 tRNA genes, that are redundant and many of them lack any 5′ regulatory sequences (e.g., Thomann et al. 1989 J Mol Biol 209:505-523).

A database of tRNA gene compilations can be found at http://rna.wustl.edu/tRNAdb/ showing that the human genome encodes 648 tRNA genes, some of them known to be pseudogenes but a majority (496) encoding functional tRNAs (many redundantly) that are needed to encode the 20 amino acids. Some tRNA genes (see http://rna wustl.edu/tRNAdb/) encode an intron located 1 base pair after the anti-codon triplet sequence.

FIG. 2 illustrates a tRNA gene and its expression and maturation into an intact tRNA molecule. A tRNA gene with its immediate 5′ and 3′ flanking sequences is shown with an intron that, in certain genes (including mammalian tRNA^{Tyr} and tRNA^{Ser} genes) is located 1 base pair (bp) downstream of coding sequence of the 3 bp anticodon. RNA polymerase III start and termination motifs are shown as “Purine(s)” in the 5′ flanking sequence (usually 4-20 bp upstream of the structural gene) and as “TTTTn” in the 3′ flanking sequence (usually 5-25 bp downstream of the structural gene). Transcription normally starts 10-30 bp upstream (5′) at a purine base and stops at an oligo-T sequence (four or more) some 5-50 bp downstream (3′) of the gene. After transcription, the pre-tRNA folds into the cloverleaf (secondary structure, see FIG. 1) and L-shape (tertiary structure). It remains in the nucleus until processing at its 5′ and 3′ ends and splicing (intron removal) has occurred (Kuwabara et al. 2001 Biomacromolecules 2:1229-1242; Dahlberg & Lund 1998 Curr Opin Cell Biol 10:400-408; Lund & Dahlberg 1998 Science 282:2082-2085; Sarkar et al. 1999 Proc Natl Acad Sci USA 1999 96:14366-14371). This mechanism is important for the invention as it will prevent that inserted molecules such as recombinant pre-tRNA-siRNA fusion molecules are exported with out correct processing into the cytoplasm and induce a non-specific effect or an adverse host reaction. If not already encoded by the gene, a conserved CCA sequence is added at the tRNA's mature 3′ end. Only then, the tRNA is transported into the cytoplasm and is available for protein biosynthesis. As also indicated in FIG. 2, sometimes an intron is located 1 nucleotide after the anticodon triplet sequence. The pre-tRNA is recognized by 5′ and 3′ processing enzymes (e.g., RNase P and Z), that remove leader and trailer sequences by endonucleolytic cleavage, releasing the leader and trailer and a tRNA molecule with mature 5′ and 3′ ends. As mentioned, in many cases the 3′ CCA triplet is not encoded in the gene and is added after cleavage of the trailer sequence. Those pre-tRNAs that possess an intron undergo a splicing reaction that removes the intron (excision) and joins the resulting tRNA halves (ligation). Splicing can occur before or after 5′/3′ maturation depending on the organism. The cleaved intron sequences and 5′ and 3′ leader sequences do not encode any recognizable motifs that are important for the processing and splicing enzymes, thereby opening the possibility to replace them by other sequences that can play a role in vitro as well as in vivo, such as siRNA, miRNA and asRNA sequences.

In FIG. 3, the 5′ and 3′ flanking sequences as well as the intron of a tRNA gene are replaced by siRNA and/or miRNA motifs, respectively. The coding sequence for the upper strand of a siRNA is fused to the 5′ end (leader sequence) of the tRNA gene and the coding sequence of the lower or interfering strand of the siRNA is fused to the 3′ end of the tRNA gene. If the tRNA gene chosen for this fusion encodes the 3′ terminal CCA sequence, this fusion will occur adjacent to that motif. The siRNA is transcribed along with the tRNA and will appear as an extended stem due to the self-complementary nature of the leader (upper strand of siRNA) and trailer (lower strand of the siRNA). This molecule is locked in the nucleus and cannot enter the cytoplasm. Both siRNA sequences are complementary but form a bubble of 2-3 unpaired base pairs when the pre-tRNA is folded. This will ensure correct processing (cleavage) by the processing enzymes Rnase P and Z (Altman 2000 Nat Struct Biol 7:827-8; Schiffer et al. 2002 EMBO J 21:2769-77). Upon 5′ and 3′ maturation processing endoncucleases will cleave the leader and trailer sequences and release a mature, active siRNA having additional uridines at the 3′ end of the lower, inhibitory strand and the mature tRNA. As shown by Stange and Beier (1992 Eur J Biochem 210:193) and van Tol et al. (1987 EMBO J 6:35-41), the intron is removed either before or after 5′, 3′ processing, dependent on the species. More importantly, the intron sequence does not encode any recognition motifs for the splicing enzymes and thus can be replaced by other sequences, e.g., encoding siRNAs, asRNAs or miRNAs. The siRNA is base paired once transcription is finished and is believed be removed by the processing enzymes as fully functional double-stranded ribo-oligonucleotide that has a overhanging oligo-T sequence at the 3′ end of the interfering strand. Only after this maturation is complete can these molecules enter the cytoplasm.

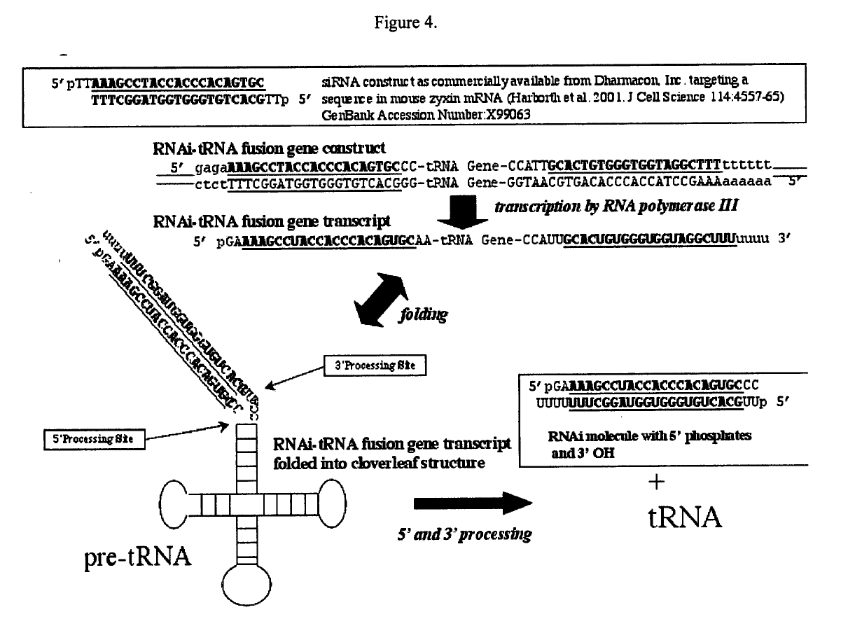

FIG. 4 shows an example of a siRNA-tRNA gene fusion vector. The sequence of the siRNA molecule that targets the mouse zyxin gene (Harborth et al. 2001 J Cell Science 114:4557-4565) can be commercially obtained from Dharmacon Inc. (Fort Collins, Colo.). The coding sequence of the upper strand of the siRNA is fused at its 3′ end to the 5′ end of the structural tRNA gene including a CC spacer sequence between the siRNA and the 5′ end of the tRNA gene. The coding sequence for the lower, inhibitory strand of the siRNA is fused at its 5′ end to the 3′ end of the tRNA gene including a CCAUU spacer between the siRNA and the tRNA gene (in this case the tRNA gene encodes the conserved 3′ CCA sequence). However, if the conserved 3′ CCA sequence is not encoded in the chosen tRNA gene it can be omitted in such a circumstance. The resulting siRNA molecule is shown after transcription of the fusion gene and correct 5′ and 3′ endonucleolytic processing has occurred. The reverse fusion; i.e., fusion of the 3′ end of the coding sequence for the lower (inhibitory) strand of the siRNA to the 5′ end of the structural tRNA gene and the 5′ end of the coding sequence for the upper strand of the siRNA to the 3′ end of the tRNA gene will also result in an active siRNA molecule after 5′ and 3′ maturation. In this case, the oligo U sequence resulting from the transcription of the transcription termination signal will be added to the 3′ end of the upper strand of the siRNA molecule instead of the 3′ end of the lower (inhibitory) strand (as shown).

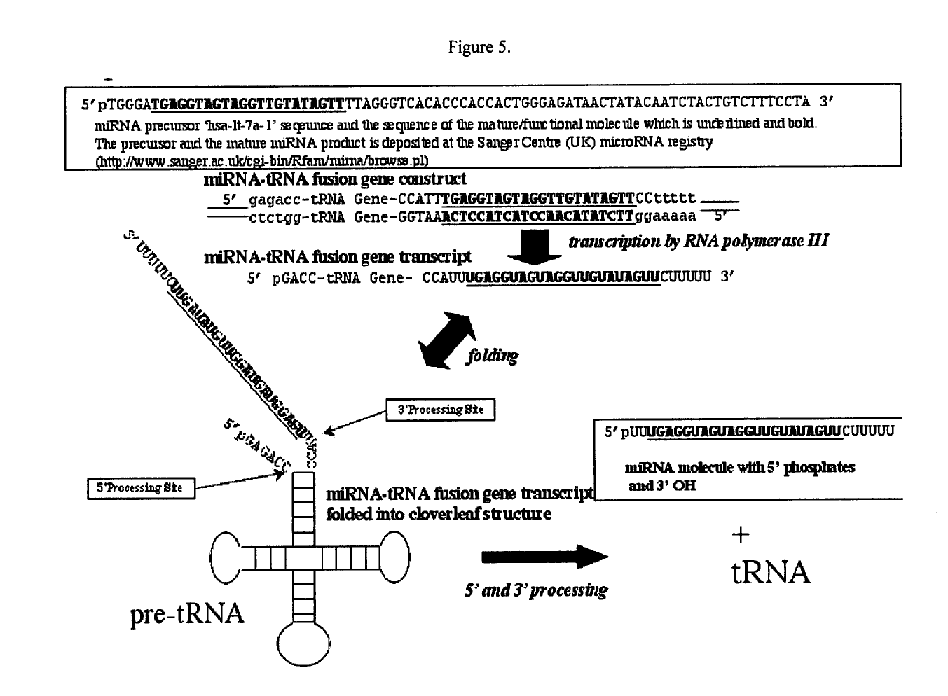

FIG. 5 shows an example of a microRNA-tRNA gene fusion vector. The precursor and mature has-lt-7a-1 miRNA sequence was deduced from a published database of known mammalian miRNAs (including human miRNAs) that are deposited at http://www.sanger.ac.uk/cgi-bin/Rfam/mima/browse.pl The example shows fusion of the 5′ end of the coding sequence for the mature miRNA to the 3′ end of a tRNA gene that encodes the conserved CCA end by adding a TT spacer. After transcription and 3′ processing the miRNA is released and can bind to its target in either nucleus or cytoplasm.

FIG. 6 shows another embodiment of a siRNA-tRNA fusion vector. This example shows the human tRNA^{Tyr} gene encoded on chromosome 14, although any other tRNA gene with an intron can principally be utilized as vector. The intron of the tRNA^{Tyr} gene is replaced by the sequence of the siRNA that targets the mouse zyxin gene (see FIG. 4.). The 5′ end of coding sequence for the upper strand of the zyxin siRNA is fused to the 3′ end of the first exon of the tRNA gene (that stops 1 bp after the GTA anticodon) using an AT spacer sequence. The 3′ end of coding sequence for the upper strand is connected to the 5′ end of the coding sequence for the lower, inhibitory strand of the zyxin siRNA via a 6 bp spacer sequence that cannot form self-complementary base pairs. Finally the 3′ end of coding sequence for the lower strand of the zyxin siRNA is fused via an AC spacer sequence to the 5′ end of the second exon of the tRNA gene. Another example is shown in form of the human miRNA sequence has-lt-7a-1 (see FIG. 5) that could also displace the native intron sequence of the tRNA gene. After transcription of the fusion gene, the pre-tRNA will fold into the two-dimensional and three dimensional structure and the leader and trailer sequences are removed (not shown), and the intron will be spliced to form a mature tRNA^{Tyr} and siRNA or miRNA (not shown). Although the siRNA is initially connected by a 6 nt loop between the 3′ end of the upper strand of the siRNA and the 5′ end of the lower (inhibitory) strand of the siRNA, such loops were shown to be processed (cleaved) in the cell to yield active siRNA (Review: Tuschl 2002 Nature Biotechnol 20:446-448). The reverse construct; i.e., by fusing the 5′ end of the coding sequence for the lower (inhibitory) strand of the siRNA to the 3′ end of the first exon of the tRNA gene, connecting the 3′ end of this strand via a 6 bp spacer with the 5′ end of the coding sequence for the upper strand of the siRNA and fusing the 3′ end of the upper strand to the 5′ end of the second exon of the tRNA gene will also result in an active siRNA molecule (not shown here). However, in this case the siRNA will initially be connected by a 6 nt loop between the 3′ end of the lower (inhibitory) strand of the siRNA and the 5′ end of the upper strand of the siRNA.

Although the invention has been set forth in detail, one skilled in the art will recognize that numerous changes and modifications can be made, and that such changes and modifications may be made without departing from the spirit and scope of the invention. The patents, patent applications and publications cited in the specification are hereby incorporated by reference herein in their entirety for all purposes.

1. A method of modulating gene expression through RNA interference comprising:
a. incorporating at least one oligonucleotide encoding a short interfering nucleic acid molecule, including coding sequence for siRNA, microRNA and/or antisense RNA into a tRNA gene construct; and
b. transforming a host cell with the gene construct of (a) under conditions wherein the short interfering nucleic acid is expressed and spliced from the expressed tRNA molecule so that the short interfering nucleic acid can enter the cytoplasm and interact with a target host molecule.

2. The method according to claim 1 wherein modulating gene expression comprises:
a. replacing all or part of an intron of a tRNA construct encoding tyrosine (tRNA^{Tyr}) or another intron-encoding tRNA gene with at least one oligonucleotide encoding a short interfering nucleic acid; and/or
b. transforming a host cell with the gene construct of (a) under conditions wherein the short interfering nucleic acid is expressed and spliced from the expressed tRNA^{Tyr} or other intron-encoding tRNA molecule so that the short interfering nucleic acid may enter the cytoplasm and interact with a target host molecule.

3. The method according to claim 1 wherein modulating gene expression comprises:
a. fusing a nucleic acid encoding the lower inhibitory or antisense strand of a double-stranded siRNA at its 5′ end to the nucleic acid encoding a tRNA gene construct at the site wherein the 3′ end of the first tRNA exon is encoded;
b. connecting the nucleic acid encoding the lower antisense strand of the siRNA at its 3′ end with nucleic acid encoding a spacer of at least one base pair to the 5′ end of nucleic acid encoding the upper sense strand of the siRNA;
c. fusing nucleic acid encoding the upper strand sense of the siRNA at its 3′ end to the 5′ end of nucleic acid encoding the second exon of the tRNA;
d. cloning the resulting modified gene construct into a linear or plasmid vector; and
e. transforming a host cell with the modified gene construct under conditions wherein the short interfering nucleic acid is expressed and spliced from the modified tRNA construct or expressed tRNA from the transcribed modified tRNA construct so that the short interfering nucleic acid can enter the cytoplasm and interact with a target host molecule.

4. The method according to claim 1 wherein modulating gene expression comprises:
a. fusing a nucleic acid encoding the upper sense strand of a double-stranded siRNA at its 5′ end to the nucleic acid encoding a tRNA gene construct at the site wherein the 3′ end of the first tRNA exon is encoded;
b. connecting the nucleic acid encoding the upper sense strand of the siRNA at its 3′ end with nucleic acid encoding a spacer of at least one base pair to the 5′ end of nucleic acid encoding the lower inhibitory or antisense strand of the siRNA;
c. fusing nucleic acid encoding the lower inhibitory or antisense strand of the siRNA at its 3′ end to the 5′ end of nucleic acid encoding the second exon of the tRNA;
d. cloning the resulting modified gene construct into a linear or plasmid vector; and
e. transforming a host cell with the modified gene construct under conditions wherein the short interfering nucleic acid is expressed and spliced from the modified tRNA construct or expressed tRNA from the transcribed modified tRNA construct so that the short interfering nucleic acid can enter the cytoplasm and interact with a target host molecule.

5. The method according to claim 1, wherein the short interfering nucleic acid molecule comprises two independent RNAi molecules.

6. The method according to claim 1, wherein the short interfering nucleic acid molecule comprises three independent RNAi molecules.

7. The method according to claim 1 wherein the host cell is a mouse, rat, primate or human cell or any animal or a plant cell.

8. A short interfering nucleic acid molecule, comprising a coding sequence for siRNA, miRNA or antisense RNA incorporated into a tRNA gene construct.

9. The short interfering nucleic acid molecule of claim 8 wherein the coding sequence is incorporated into a tRNA gene construct by fusion of the 3′ end of the coding sequence to the 5′ end of the tRNA gene.

10. The short interfering nucleic acid molecule of claim 8 wherein the coding sequence is incorporated into a tRNA gene construct by fusion of the 5′ end of the coding sequence to the 3′ end of the nucleic acid encoding the tRNA gene.

11. The short interfering nucleic acid molecule of claim 8 wherein the coding sequence is incorporated into a tRNA gene construct by fusion of the 5′ end of the coding sequence to the 3′ end of the nucleic acid encoding the first exon of tRNA gene and fusion of the 3′ end of the coding sequence to the 5′ end of the nucleic acid encoding the second exon of the tRNA gene.

12. The short interfering nucleic acid molecule of claim 8 wherein the coding sequence encodes 19-30 nucleotides of a sense strand of a siRNA and 19-30 nucleotides of an antisense strand of the siRNA.

13. The use of a naturally-occurring or synthetic tRNA gene construct for the manufacture of a pharmaceutical composition for delivery of a therapeutic interfering nucleic acid molecule into an organism, wherein the interfering nucleic acid molecule is selected from the group consisting of a siRNAi, miRNA and antisense RNA.

14. The use of the pharmaceutical composition according to claim 13 for the treatment of cancer.

15. The use of the pharmaceutical composition according to claim 13 for the treatment of genetic diseases.

16. The use of the pharmaceutical composition according to claim 13 for the treatment of inflammation or pain.

17. A kit for transfecting a cell wherein the kit comprises nucleic acid encoding a short interfering RNA incorporated into a tRNA vector.

18. A kit according to claim 17 wherein the kit comprises nucleic acid encoding an antisense RNA incorporated into a tRNA vector.

19. A method of treating an organism comprising administering a tRNA gene construct modified to incorporate nucleic acid encoding a short interfering RNA, microRNA or antisense RNA.

20. The method of claim 19 wherein the organism is a mouse, rat, primate or human or any animal or a plant.
